(19) United States
(12) Patent Application Publication
(10) Pub. No.: US 2002/0018817 A1
(43) Pub. Date: Feb. 14, 2002

(54) Method for treatment of asthma syndrome

(75) Inventors: Ba X. Hoang, San Jose, CA (US); Stephen A. Levine, Kentfield, CA (US)

Correspondence Address: PERKINS COIE LLP, P.O. BOX 2168, MENLO PARK, CA, 94026, US

(73) Assignee: Scottsdale Scientific DBA Allergy Research Group/ Nutricology, Inc.

(21) Appl. No.: 09/954,859

(22) Filed: Sep. 17, 2001

Related U.S. Application Data
(63) Continuation of application No. 09/611,036, filed on Jul. 6, 2000, now abandoned.
(60) Provisional application No. 60/142,482, filed on Jul. 6, 1999.

(51) Int. Cl. A61K 35/50

(52) U.S. Cl. 424/583

(57) ABSTRACT

A method of treating asthma in a patient is disclosed. The method includes administering to the patient, a therapeutically effective amount of (i) Moschus moschiferus or its active ingredient muscone, (ii) Calculus Bovis or its major active ingredients cholic acid and urodesoxychloic acid, or (iii) Placental extract or powder from humans or mammals. The compound is preferably administered orally, twice or three time a day.

This application is a continuation of U.S. application Ser. No. 09/611,036 filed Jul. 6, 2000, now pending; which claims the priority of U.S. Provisional Application No. 60/142,482 filed Jul. 6, 1999, which are incorporated herein by reference in their entirety.

### BACKGROUND OF THE INVENTION

Despite developments in understanding the nature of asthma and developing new treatment procedures, the occurrence of asthma has increased significantly over the last 20 years. Hospital admissions for asthma treatment have increased and deaths attributed to asthma have doubled since 1976. Today, nearly 20 million Americans suffer from asthma, of which about 5 million are children.

Asthma is traditionally divided to: allergic (extrinsic) and non-allergic (intrinsic) types of asthma. However, this classification has no impact in selecting a therapy for acute asthma attacks, in preventive treatments or in prognosis of asthma severity. Treatments of asthma are the same for both types of asthma. Physicians try to treat every symptom of asthma and make adjustments or changes in asthma therapy. But these adjustments are based on patients'responses to the various drugs without comprehensive understanding or explanation. The uniform therapeutic strategy and unpredictable responses of the patients to different therapeutic agents represent a formidable problem for asthma treatments as well as preventive therapy.

Currently, there are six major classes of asthma medications: (i) steroidal anti-inflammatories, (ii) allergy-blocking agents (cromolyn sodium and nedocromil sodium), (iii) beta-agonists; (iv) xanthines (theophylline); (v) anti-cholinenergics; and (vi) anti-leukotrienes. Among these, glucocorticosteroids are the most effective therapy available for asthma. Therapy with inhaled glucocorticosteroids is now recommended at a much earlier stage. However, despite their proven efficacy in the treatment of asthma, enthusiasm for their use has been gradual due to reported systemic side effects and the extensive periods of use by patients such as children. Because of this concern, less effective therapies are often preferred. Cromolyn sodium and nedocromil sodium are less effective in controlling asthma than inhaled glucocorticoids. They need to be taken four times daily and although they may be useful for some patients with mild asthma, their efficacy often decreases in long-term therapy.

Conventional beta 2-agonists do not control asthma as effectively as inhaled glucocorticoids in either adults or children. The long-acting inhaled beta-2 agonist was currently recommended to be added to the inhaled steroid group. Theophylline alone does not control asthma as effectively as inhaled glucocorticosteroids and produces more side effects. However, there is a group of patients who responded only to theophylline and beta-2 agonist but not to inhaled steroids.

Most recently added to the complexity of therapeutic measures of asthma is a new class of preventive medications called anti-leukotrient agents: accolate, zileuton and zyflo. Studies show that they can improve symptoms substantially for ⅓ of treated patients, particularly when combined with all other drugs. For the other ⅓ of the treated patients, there are only minimal improvements. This class of medication has no impact on the last ⅓ of the treated patients. The side effects of anti-leukotrients can be quite serious and they are not recommended for use in lifelong treatments.

Immunotherapy is one of the therapeutic methods which was recommended for patients with an allergic type of asthma. However, some recent studies of using immunotherapy with injections of allergens for over two years was of no discernible benefit in allergic children (NEJM Jan. 30, 1997—Volume 336, Number 5).

As the controversy and unpredictability in pharmacological therapy for asthma continues, patients with moderate and severe asthma are treated with all available classes of the mentioned drugs. These drugs offer symptom control and only recommended as long as they are effective. The discontinuance of the therapy usually leads to a severe rebound of asthma symptoms. Although these old and new drugs offer some improvements in asthma management, the adverse asthma outcomes (incubation, respiratory distress and death) among hospitalized patients are increasing (Daphne Calmes MD, MSHS: Adverse Asthma Outcomes among Children Hospitalized With Asthma in California, Pediatrics, Vol. 1015 May 1998 pp 845-850). There is also a group of patients who responded only to systemic steroids with abundant side effects and life-threatening asthma attacks. Currently, there are perspective therapies on the line for these severe patients.

### SUMMARY OF THE INVENTION

In accordance with the present invention, it has been discovered that several natural agents, including Moschus moschiferus (the major active component of which is muscone C₁₆H₃₀O, also 3-methylcyclopentadecanone), Calculus Bovis or animal bile concentrate (the major active components of which are cholic acid, C₂₄H₄₀O₅ and ursodeoxycholic acid C₂₄H₄₀O₄) and placental extract or powder from humans or mammals.

These therapeutic agents are administered in a therapeutically effective amount to a patient specifically for the following types of types of asthma attacks:

Type 1: Hot or Red Type

The main clinical feature are red or rose face, swelling rose or salmon color of the tongue. The therapeutic agent for this type of asthma is Calculus bovis, or cholic acid and/or ursodeoxycholic acid, given in oral dose of about 0.05 to 1 g every 8 hours, preferably about 0.05 to 0.2 grams, e.g., 0.1 gram/adult/8 hours.

Type 2: Cold or Pale Type

The main clinical feature are pale or white face, constricted and pale tongue. The therapeutic agent for this type of asthma is Moschus moschiferus, or muscone, given in oral dose of about 0.05 to 1 g every 8 hours, preferably about 0.05 to 0.2 grams, e.g., 0.1 gram/adult/8 hours.

Type 3: Infection Related Asthma

The asthma attacks are associated with clinical symptoms of lung or bronchial infections. The therapeutic agent for this type of asthma is placenta extract or powder, given in oral dose of about 1 to 10 grams every twelve hours, preferably about 1 to 4 grams, e.g., 2 gram/adult/8 hours.

In using conventional medications, it is usually more effective to use either bronchodialators, adreno-agonists or theuphylline preparation for type 2 and type 3 of asthma. Type 1 of asthma is more responsive to anti-inflammatory preparation such as oral or IV cortico-steroidal preparation.

### DETAILED DESCRIPTION OF THE INVENTION

The following treating methods were employed in treating asthma, in accordance with the present invention.

Type 1: Hot or Hyperactive

Participants:

Eighteen patients, ages 3 to 68 years who were being treated for an acute asthma exacerbation with peak expiratory flow rate (PEFR) had less than 60% of the predicted value after receiving 2-3 beta 2-adrenergic inhalers. All patients experienced the typical and severe physical symptoms of asthma attacks.

Interventions:

Calculus bovis in the dosage of 0.1 grams were given orally every 8 hours.

Initial significant relief of symptoms and lung function (PEFR) followed within 2-5 minutes after administration of the treatment. After 1-3 days, all patients were symptom-free and normal breathing functions were restored. All patients reported that they had never experienced the complete relief and well-being feeling as they had received with the applied therapy. No adverse effects were reported. No patient had a significant change in blood pressure or other vital functional parameters. All patients were able to completely avoid bronchodialator therapy or reduced 90% of it's use. No patients needed hospitalization or professional medical care.

Type 2: Cold or Hyperactive Type

Participants:

Twenty-one patients ages 13-62 years old were being treated for acute asthma attacks. These attacks were resistant to 2-3 bronchodialator therapy and oral theophylline preparation. The (PEFR) were less than 60% of the predicted value. Ten patients suffered excessive mucus production, which was also resistant to all conventional therapeutic methods.

Interventions:

Moschus moschiferus was given in the dosage of 0.1 grams orally every 8 hours.

The first significant improvement in both subjective and functional parameters were followed in 2-3 minutes. Within three days of therapy, the vast majority of the patients treated were completely symptom-free with lung functions improving 90-100% when compared to baseline (normal) data.

There were no side effects reported. There was a significant improvement of mucus production and subjective well-being of the patients.

Type III—Infection Related

Participants:

Fifteen patients ages 4-56 years who suffered from asthma with signs of low resistance to exercise, cold weather and pathologic agents. They exhibited no significant allergic history although recurring infections were common. These patients developed long-lasting asthma active episodes which required treatments by complex drugs such as bronchodialators, oral or steroid inhaler, theophylline and anti-biotics.

Intervention:

Patients were treated with 2 grams of placenta hominis in a powdered form orally every 12 hours. The remarkable improvements occurred within 30 minutes to 2 hours. The patients had eliminated all symptomatic drugs for both the asthma and the infections. Asthma and infectious symptoms were extinct within three days after therapy. Patients experienced significant improvement in their health and well-being. No known side effects have resulted from this type of therapy.

Some of the most impressive features of the proposed medicines are prompt action and simple application methods; orally or sublingually (at home without the need of professional ER care). They are suitable for treating unconscious patients as well.

There is not a remission period or any dependency on these medicines which is common in traditional drugs.

The implementation of the proposed medicines will substantially improve asthma management, reduce the severity of asthma attacks, hospitalization and death rates.

Therapy for Chronic Asthma (Adults and Children)

1—Therapy for Hot or Hypereactive Type (Type 1)

It has been discovered that one natural agent: animal bile from Hogs, Bovines, Rabbits and Crocodiles (the main active compound is cholic acid).

Results: Participants:

12 patients with type 1 asthma ages 2-48 years old have been involved with the therapy, but with unsatisfactory results using conventional treatments. Nine patients had a history of allergic disorders and no signs of infection or physical problems. Other associated disorders include stress, anxiety, irritability, rose or salmon colored and swelling tongue. Conventional treatments for these patients involved a complex combination of beta 2-agonist long-acting, steroidal inhalers, Chromone Salts, Theo-dur, Leukotrients antagonists. Despite heavy amounts of treatment, the patient's health was significantly hampered due to excessive medication and continual asthma symptoms.

Intervention:

patients were then treated with 1 g of hog or crocodile bile with 5 grams of natural bee honey twice a day. The patients all responded well and immediately. After 3-10 days of therapy, the asthma symptoms and the need of bronchodialator therapy were reduced to 80-100%. The patients were then able to decrease their preventive medication such as: steroid inhalers, Chromone salts inhaler, Theo-dur and Leukotrien-antagonist gradually. In 2 to 6 weeks, they were able to avoid it completely. No adverse reactions or intolerance had occurred during the course of therapy or after one year later.

2—Therapy for Cold—Hyperactive (Type II)

Participants:

Eighteen patients with moderate and severe asthma clinical symptoms who were resistant to traditional therapy including steroid inhalers, short and long acting beta 2-agonist, and Theophyline. These patients exhibited a loss of color in the face and looked pale as well as having a pale and constricted tongue. They often experienced reduced reaction time, a weakness in their voice and physical activity, and a lack of desire to drink. Intervention: Patients were treated with a combination of 0.05 grams of Moschus Moschiferus and 2 grams Cordyceps Sinesis (or crocodile meat or sea horse) daily. The patients experienced a major improvement in their quality of life. Asthma symptoms and the use of bronchodialator inhaler was significantly reduced within three days. After 2-6 weeks, the patients were able to completely avoid all symptomatic and preventive drugs and no longer suffered from any asthma symptoms or physical restriction. We recommended the patient to continue treatment for 3 months and to maintain therapy with Cordyceps Sinesis (or crocodile meat or see-horse).

There were no side effects or intolerance from the therapy using the previous mentioned agents during a course of treatment of up to one year.

3—Therapy for Infection-dependent or Low Resistant Type of Asthma

Results: Participant:

Sixteen patients ages 4-56 years olds have been involved with our therapy due to the lack of satisfactory results from traditional drugs including: Beta-2-agonists, Theophylline, Steroids inhalers, Leukotrient-inhibitors, and Antibiotics. Before the arrival of typical asthma symptoms, all patients experienced shortness of breath, frequent respiratory infections, bronchitis, and sinusitis which were sometimes resistant to anti-bacterial therapy.

Interventions:

These patients were placed on 2 grams of placenta hominis powder twice a day. Their conditions improved dramatically in the first day of therapy. They no longer experienced breathing difficulties or asthma and infection symptoms after 2-3 days of therapy. They were able to stop using all their symptomatic and preventive medications without any relapse of symptoms. They also experienced significant improvement in quality of life, weight control, physical activity blood pressure (some patients with this type of asthma developed this) and associated hypertension. The patients were also resistant to viral and bacterial infections.

Patients undergoing this treatment were now able to enjoy foods that they could not consume before without any problems. They have no symptomatic restraint such as walking or other physical exercises which was present during conventional treatment. Even when the symptoms were considered completely controlled, some chest congestion still existed with conventional medication. In addition, the majority of the treated patients are now able to tolerate triggering factors such as dust mites or cat-dog dander, physical exercise or viral infection without developing asthma attacks. In other words, the proposed approach leads to complete restoration of non-asthmatic status for the majority of treated patients rather than offer band-aid relief for asthma symptoms.

The significant advantage of our therapeutic approach is that the treatment does not lead to drug-dependency or rebound phenomena. Patients experienced a continual decreasing need in both their dosage level and the frequency of therapy, while experiencing a continual improvement in the reduction of all related symptoms.

Since most patients remain in the same environment, they are continually exposed to the same environmental factors which originally induced the reactions. Therefore we expect that some patients may experience a reoccurrence, which is easily and quickly controlled by using the same therapy.

All in all, we have refined and developed this therapeutic program after 40 years of research by incorporating our knowledge of both Western and Eastern Medicines. We have refined the experience of hundreds of Oriental specialist who have successfully treated asthma with various natural products and breathing management. These are some of the key breakthroughs in developing our treatments:

1. The therapeutic program for asthma is approached from very different perspectives other than the conventional theory. Here asthma symptoms are defined in terms of defensive reactions of the body to some functional and metabolic imbalances.

2. It is has been discovered that asthma is a clinical-functional syndrome with various causes. It has been discovered three major clinical types of asthma syndrome and three different specific therapeutic agents for each type of asthma,

3. The natural therapeutic agents for treatments of asthma attacks and chronic asthma will have no significant side effects or direct actions toward each symptom of asthma and offer fast and complete relief for the patients. Moreover, it is likely that by resolving the underlying causes, these therapeutic agents not only control asthma symptoms but also promote overall well-being of the patients, relieve associate health problems, and improve patient's resistance to environmental and pathologic agents.9

4. One of the most distinguished advantage of our therapeutic approach is it's potential ability to promote a cure for some patients. It has no side effects, rebound or dependency phenomena.

All asthma basic and applied research including research studies in the field of new drug discovery usually performs on animal disease models, gene or cell-or molecular screening assays. These models and assays display only each symptom or pathogenesis compartment of asthma, but not the underlying pathologic process which is what really occurs in the patients. The common trend in asthma research does not discriminate the principle differences between asthma and other allergic, inflammatory diseases or between patients with different types of asthma. In contrast, our therapeutic approach has been developed through clinical practice dealing directly with the patient's life and their diseases.

Physicians who experienced asthma management noticed remarkable differences in asthma clinical course and responses to therapy. They all agreed that different types of asthma may require different therapies. However, the therapeutic direction previously, currently and in future avenues are dictated by basic researchers. Geneticists, Pharmacologists and immunologists considered asthma as one disease. Each tries to identify one gene, one mediator, one pathogenesis pathway which produces asthma symptoms in order to create the inhibitors for these factors as the possible treatments for asthma.

The main therapeutic directions are: T lymphocytes, Eosinophils, Pathway antagonists, Adhesion molecules and Gene therapy.

Since asthma is poorly defined and continues to defy description and if asthma is correctly characterized as a syndrome rather than a disease, the search for optimal treatment or cure will become complex. The causes of the component disorders of the asthma syndrome may result in the need for multiple therapies.

Asthma is also unlikely to be amenable to gene therapy, even after advances are made with less complex disorders. The polygenomic nature of the syndrome and the inability to define a specific pathogeneticprocess link to a final common pathway suggests that gene therapies will probably not be feasible in the near future.

Novel pharmaceutical approaches, although exciting, are associated with a number of potential risks. The double-edged sword of therapy that directly or indirectly suppresses some immune or vital function of the body in order to suppress asthma symptoms must be weighed carefully when being considered for the treatment of what is historically a benign disease.

In contrast to the general trend in asthma research and therapy, we considered that all asthmatic symptoms are defensive reactions of the body to low natural anti-oxidant activity of liver system in type 1 (Hot type), Neuro-thermodynamic deficiency in type 2 (cold type) and bronchial immunodeficiency in type 3 (infection and low resistant type). By promoting the restoration of the mentioned imbalances or deficiencies, the proposed natural products help to eliminate the asthma symptoms effectively and safely. In some cases, even a cure is offered for individuals with asthma syndrome.

It is claimed:

1. A method of treating asthma in a patient, comprising
administering to the patient, a therapeutically effective amount of a composition selected from the group consisting of Moschus moschiferus or its active ingredient muscone, Calculus Bovis or its major active ingredients cholic acid and urodesoxychloic acid, and Placental extract or powder from humans or mammals.

2. The method of claim 1, wherein the composition is administered orally or sublingually.

3. The method of claim 2, wherein the composition contains muscone as its major active ingredient, and said administering is effective to administer muscone in an amount of between about 0.05 to 0.25 grams/patient every twelve hours.

4. The method of claim 2, wherein the composition contains cholic acid as its major active ingredient, and said administering is effective to administer cholic acid in an amount of between about 0.05 to 0.25 grams/patient every twelve hours.

5. The method of claim 2, wherein the composition contains ursodeoxycholic as its major active ingredient, and said administering is effective to administer ursodeoxycholic acid in an amount of between about 0.05 to 0.25 grams/patient every twelve hours.
